(19) United States
(12) Patent Application Publication
(10) Pub. No.: US 2025/0297962 A1
(43) Pub. Date: Sep. 25, 2025

(54) DEVICES, METHODS, AND SYSTEMS TO MEASURING AND RECORDING SPECTRUM OF A REACTANT ARRAY

(71) Applicant: SENSILL, INC., Concord, CA (US)

(72) Inventors: Yan Zhou, Pleasanton, CA (US); Richard Wyeth, Granite Bay, CA (US); William Shea, Martinez, CA (US)

(73) Assignee: SENSILL, INC., Concord, CA (US)

(21) Appl. No.: 19/229,714

(22) Filed: Jun. 5, 2025

Related U.S. Application Data
(63) Continuation of application No. PCT/US2023/083063, filed on Dec. 8, 2023.

(51) Int. Cl. G01N 21/78; G01N 21/77; G01N 31/22

(52) U.S. Cl. CPC G01N 21/783; G01N 21/7703; G01N 31/22; G01N 2021/7726; G01N 2201/0638

(57) ABSTRACT

Devices, systems, and methods include a system comprising a substrate having a transparent portion, one or more reactants on a first surface of the transparent portion of the substrate, a light source, and a light collector. The light source may be configured to illuminate the one or more reactants through the transparent portion of the substrate and the light collector may be configured to collect light from the one or more reactants. The light from the one or more reactants may be collected from the reactants without the light from the reactants traveling through the substrate and/or may be collected from the reactants after the light from the reactants has passed through the substrate.

### CROSS-REFERENCE TO RELATED APPLICATIONS

This application is a continuation of International Application No. PCT/US2023/083063, filed Dec. 8, 2023, which claims priority to: U.S. Provisional Patent Application Ser. No. 63/431,507, filed Dec. 9, 2022, the entirety of which is incorporated herein by reference; U.S. Provisional Patent Application Ser. No. 63/431,510, filed Dec. 9, 2022, the entirety of which is incorporated herein by reference; U.S. Provisional Patent Application Ser. No. 63/431,519, filed Dec. 9, 2022, the entirety of which is incorporated herein by reference; U.S. Provisional Patent Application Ser. No. 63/431,525, filed Dec. 9, 2022, the entirety of which are incorporated herein by reference; U.S. Provisional Patent Application Ser. No. 63/431,528, filed Dec. 9, 2022, the entirety of which are incorporated herein by reference; U.S. Provisional Patent Application Ser. No. 63/431,533, filed Dec. 9, 2022, the entirety of which are incorporated herein by reference.

### TECHNICAL FIELD

The present disclosure pertains to sensing and analysis tools, and the like. More particularly, the present disclosure pertains to devices and systems for sensing and analyzing chemical substances, and methods for manufacturing and using such devices.

### BACKGROUND

A wide variety of devices have been developed for collection, storing, sensing, and analysis of samples. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for sensing and analysis devices. Although it is noted that collection, storing, sensing, and analysis approaches and systems are known, there exists a need for improvement on those approaches and systems.

An example system may include a substrate, one or more reactants on the substrate, a light source, a lens focusing system, and a light collector, wherein the light source may be configured to illuminate the reactants, the lens focusing system may be configured to focus light reflecting or scattering or reemitting off of the reactants, and the light collector may be configured to collect the focused light reflecting off of the reactants.

Alternatively or additionally to any of the embodiments in this section, the substrate may be a transparent substrate.

Alternatively or additionally to any of the embodiments in this section, the substrate may have a trapezoidal cross-section.

Alternatively or additionally to any of the embodiments in this section, the substrate may have a semi-circle cross-section.

Alternatively or additionally to any of the embodiments in this section, the reactants may be applied to a first side of the substrate and the light source is configured to illuminate the reactants from a second side of the substrate.

Alternatively or additionally to any of the embodiments in this section, the lens focusing system may include a cylinder lens and an aspheric lens.

Alternatively or additionally to any of the embodiments in this section, the light collector may be an optical fiber.

In another examples, a system may include a substrate having a transparent portion, one or more reactants on a first surface of the transparent portion of the substrate, a light source, and a light collector, wherein the light source may be configured to illuminate the one or more reactants through the transparent portion of the substrate and the light collector is configured to collect light from the one or more reactants.

Alternatively or additionally to any of the embodiments in this section, the transparent portion of the substrate may extend between the first surface and a second surface of the substrate, wherein the second surface of the substrate is non-parallel with the first surface.

Alternatively or additionally to any of the embodiments in this section, the light source may be configured to illuminate the one or more reactants by applying light to the one or more reactants through the second surface.

Alternatively or additionally to any of the embodiments in this section, the second surface may be rounded.

Alternatively or additionally to any of the embodiments in this section, the system may further include a lens positioned between the light source and the second surface, wherein the lens may be configured to focus light from the light source on a reactant of the one or more reactants.

Alternatively or additionally to any of the embodiments in this section, a cross-section of the substrate may be symmetrical about a center line extending perpendicularly through the first surface.

Alternatively or additionally to any of the embodiments in this section, a shape of a cross-section of the substrate may be selected from a group consisting of a trapezoid, a semi-circle, and a triangle.

Alternatively or additionally to any of the embodiments in this section, the system may further include a collection lens system, wherein the collection lens system may be configured to focus light from the one or more reactants for collection by the light collector.

Alternatively or additionally to any of the embodiments in this section, the collection lens system may include a cylinder lens and an aspheric lens.

Alternatively or additionally to any of the embodiments in this section, the system may further include an optical fiber configured to collect light from the one or more reactants.

Alternatively or additionally to any of the embodiments in this section, the light collector may comprise one or more light sensors configured to receive light from the one or more reactants and the one or more light sensors are selected from a group consisting of a spectrometer, a contact imaging sensor, a camera, and an n-dimensional sensor array.

Alternatively or additionally to any of the embodiments in this section, the system may further include a layer of porous material on the first surface of the substrate, wherein the one or more reactants may be on the layer of porous material.

In another example, a meth may include applying light through one or more transparent portions of a substrate to one or more reactants on the substrate, collecting light from the one or more reactants, and measuring levels of a wavelength of the light from the one or more reactants.

Alternatively or additionally to any of the embodiments in this section, the method may further include exposing the one or more reactants to a fluid and identifying a component of the fluid based on the levels of the wavelength of the light from the one or more reactants.

Alternatively or additionally to any of the embodiments in this section, the collecting light from the one or more reactants may include collecting light from the one or more reactants through the one or more transparent portions of the substrate.

Alternatively or additionally to any of the embodiments in this section, the applying the light through the one or more transparent portions of the substrate to the one or more reactants may include focusing the light on a reactant of the one or more reactants.

Alternatively or additionally to any of the embodiments in this section, a transparent portion of the one or more transparent portions of the substrate may extend between a first surface on which the one or more reactants are located and a second surface of the substrate, wherein the second surface of the substrate is non-parallel with the first surface.

In another example, a colorimetric sensor array may include a substrate having a transparent portion and one or more reactants on a first surface of the transparent portion the substrate, wherein the transparent portion of the substrate may extend between the first surface on which the one or more reactants are located and a second surface of the substrate, wherein the second surface of the substrate is non-parallel with the first surface.

Alternatively or additionally to any of the embodiments in this section, the second surface may be rounded.

Alternatively or additionally to any of the embodiments in this section, a cross-section of the substrate may be symmetrical about a center line extending perpendicularly through the first surface.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative sensing system;

FIG. 2 is a schematic diagram of an illustrative sensing system;

FIG. 3 is a schematic diagram of an illustrative computing system;

FIG. 4 is a schematic perspective view of an illustrative sensing system;

FIG. 5 is a schematic front view of an illustrative sensing system;

FIG. 6 is a schematic front view of an illustrative sensing system;

FIG. 7 is a schematic perspective view of an illustrative colorimetric sensor array;

FIG. 8 is a schematic perspective view of an illustrative colorimetric sensor array;

FIGS. 9A and 9B depict schematic side and front views, respectively, of an illustrative sensing system;

FIGS. 10A and 10B depict schematic side and front views, respectively of an illustrative sensing system; and

FIG. 11 depicts a schematic diagram of an illustrative technique for using a sensing system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “fluid” is inclusive of both liquids and gases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/of” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Fluids with concentrations of volatile compounds (e.g., volatile organic compounds (VOCs)) and/or gasses, which may or may not be hazardous, may be sensed, analyzed, and/or monitored. Sensing, analyzing, and/or monitoring of fluids with analytes (e.g., non-volatile and/or volatile compounds, gases, liquids, and/or other fluids) may utilize absorption measurements of reactants exposed to such fluids for any purpose including, but not limited to, diagnostic hazard warning, manufacturing processes or quality control, record keeping, archival purposes, product development, product-consumer matching, etc.

In some cases, VOCs and/or gasses may be present in ambient fluid (e.g., ambient air, etc.) and sensed, analyzed, and/or monitored using reactants for real-time alarms, to treat subjects, or to collect and/or archive data for health records, regulatory compliance records, etc. Further, VOCs and/or gasses exhaled or emitted, excreted, emanated, released, and/or secreted from a subject (e.g., humans, animals other than humans, food, produce, meat, pathogens, bacteria (e.g., good and/or bad bacteria), plants, wounds, ulcers, surgical sites, skin of a subject, mouth of a subject, nasal passages of a subject, sinuses of a subject, rectum area of a subject, vaginal area of a subject, genitals area of a subject, ear canals of a subject, pores of a subject, etc.) may be sensed, analyzed, and/or monitored to assess hazardous, dangerous, or illegal substances in or at the subject or target site, a lung condition of lungs of a subject, a condition of a blood disease, a condition of infections, conditions related to diseases or biological conditions, conditions related to general health, conditions related to food flavors, conditions related to perfumes or smells, and/or other suitable conditions.

The systems discussed herein for sensing, analyzing, and/or monitoring fluids (e.g., for analytes of interest) may be configured to accurately detect and record a colorimetric sensor array (CSA) spectral response to exposure to the fluids. The systems may utilize techniques for non-invasively detecting one or more analytes of interest (e.g., one or more pathogens responsible for specific human skin infections including, but not limited to, skin infections, urinary tract infections (UTIs), vaginitis, wound infections, ulcers, etc., and/or other suitable analytes) from a fluid using a CSA to allow for early detection of and early implementation of protocols to address one or more conditions associated with any sensed analytes of interest. In one example, enhanced classification of one or more analytes using the systems described herein may enable detection and identification of responsible pathogens at the very beginning stages of a dangerous skin infection, which may result in a high level of protection and probability of a favorable outcome for subjects.

The systems for sensing, analyzing, and/or monitoring analytes of fluids may use optics to capture photons diffused, reflected, scattered, transmitted, or reemitted from individual reactants (e.g., color areas, color imprints, color bars, color dots, etc.) applied to a substrate or membrane of a CSA and deliver the photons via a fiber optic cable or free space optics to a light collector (e.g., a high-resolution spectrometer having a photodetector and/or other suitable light collector) for measurement of collected light. Appropriate calibration techniques and an algebraic signal processing algorithm may be applied to the measurements to calculate a light collection measurement (e.g., reflectivity, intensity, pixel value, photon count, etc.) This technique may be applicable for wavelengths extending from the ultra-violet, through the visible, and into the mid-infrared portion of the spectrum. In some cases, a motion stage (e.g., an adjustable stage) may be employed to facilitate collecting multiple spectra at discrete locations over a full reactant array of the CSA.

The systems for sensing, analyzing, and/or monitoring components of a fluid (e.g., for analytes of interest, etc.) may capture and process data iteratively or continuously on-the-fly as the entire reactant array or an entirety of a portion of the reactant array of the CSA is viewed for processing. The captured or obtained data (e.g., spectral data, etc.) may then be processed to accurately associate the captured or obtained data with each reactant in the CSA. During a single fluid analysis test, the reactant array or a portion of the reactant array may be viewed for processing one or more times. By performing repetitive measurements over time, the changes to the collected spectra of some or all reactants of a reactant array (e.g., reactant array of a CSA) may be recorded during exposure of the reactant array to a fluid and used to identify components of the fluid (e.g., analytes of interest).

The systems for sensing, analyzing, and/or monitoring analytes may facilitate enhancing an efficiency of the systems in terms of illuminating reactants of the CSA and collecting diffused, reflected, scattered, transmitted, or reemitted light from the reactants via an optical fiber that is coupled with a spectrometer. To facilitate enhancing the efficiency of the systems, illumination may be launched and focused from below a CSA of the system via a transparent substrate (e.g., a substrate having at least a transparent portion) of a desired size and/or configuration onto reactants in such a manner that no or minimal direct illumination light may be collected by a diffused light collection optical design. In such configured systems with focused light applied to the reactants from below the reactants, potential optical and/or mechanical interference that can limit diffused light collection efficiency of the system when light is applied to the reactants from above is removed.

To focus the light from the reactants, the systems for sensing, analyzing, and/or monitoring analytes may include a cylinder lens and a sphere or aspheric condensing lens to optically relay or guide light rays from an area having a first shape or configuration (e.g., a shape having a same area as a reactant, such as a line or rectangular area having a same or similar area as a color bar or within the area of a color bar, etc.) to an area having a second shape (e.g., a shape having a same area as a light collecting element, such as a circular spot area for light collection by an optical fiber (e.g., a multimode optical fiber or other suitable optical fiber or free space light collection optics)). The focusing of the light may result in a natural averaging along each reactant and as a result, scanning to collect information from each reactant of an array may only need to occur along a multiple reactant direction.

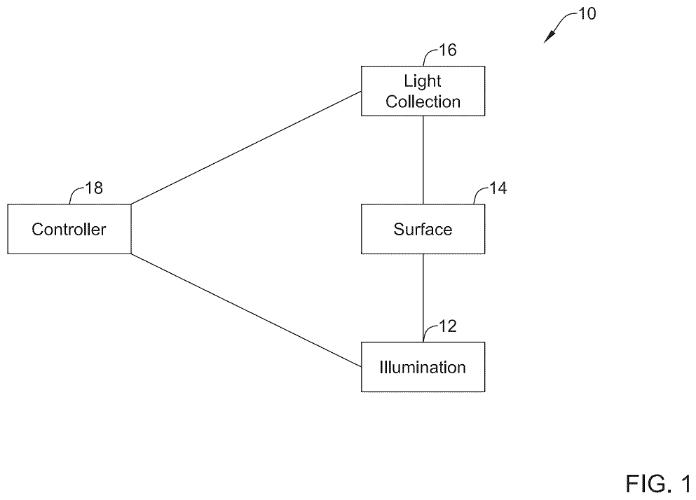

Turning to the Figures, FIG. 1 schematically depicts an illustrative configuration of a fluid analysis system 10 for determining a component of a fluid. In some examples, the fluid analysis system 10 may include, among other components, an illumination component 12 configured to illuminate one or more reactants (e.g., an analyte sensitive material) of a reactant array on or otherwise supported by a surface 14, a light collection component 16 configured to receive or collect light from the one or more reactants, and a controller 18 configured to be in communication with the illumination component 12 and/or the light collection component 16. In some examples, the illumination component 12 and/or the light collection component 16 may form or be part of an optical system of the fluid analysis system 10. The controller 18 may be configured to analyze or facilitate analyzing data related to light collected at the light collection component 16.

The one or more reactants of the reactant array on or supported by the surface 14 may be exposed to fluid. In some examples, the one or more reactants may be exposed to fluid in any suitable manner including, but not limited to, by pumping fluid to or along the one or more reactants during a fluid test using the fluid analysis system 10, exposing the one or more reactants to the fluid prior to being positioned in the fluid analysis system 10, positioning the one or more reactants proximate an area of interest (e.g., a wound, etc.) prior to being positioned in the fluid analysis system, and/or the one or more reactants may be exposed to fluid in one or more other suitable manners. Once the one or more reactants have been exposed to fluid for analysis of the fluid and light has been collected from the one or more reactants during a fluid analysis test, the controller 18 may analyze light collection data to identifying one or more components (e.g., analytes) of the fluid to which the one or more reactants were exposed.

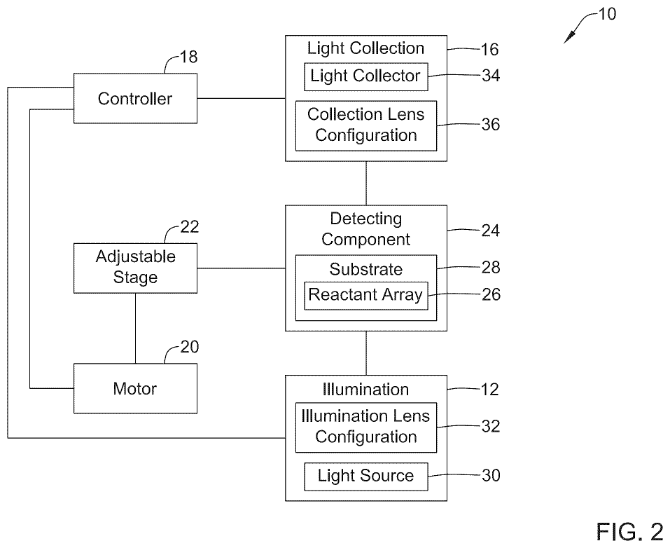

FIG. 2 schematically depicts a diagram of an illustrative configuration of the fluid analysis system 10 including the illumination component 12, the light collection component 16, and the controller 18. In some examples, the fluid analysis system 10 may additionally include a motor 20 in communication with the controller 18 and an adjustable stage 22 including or coupled with a detecting component 24 (e.g., a colorimetric sensor array (CSA) and/or other suitable detecting component). The detecting component 24 may include a reactant array 26 having the one or more reactants and a substrate 28 supporting the reactant array 26. In some cases, the substrate 28 may be or may include the surface 14 depicted in FIG. 1, but other configurations are contemplated. Optionally, the fluid analysis system 10 may include a housing configured to house one or more of the illumination component 12, the surface 14, the light collection component 16, the controller 18, the motor 20, the adjustable stage 22, the detecting component 24, and/or other suitable components of the fluid analysis system 10.

The detecting component 24 may be configured in the fluid analysis system 10 to be adjusted relative to the illumination component 12 and/or the light collection component 16 to facilitate collecting light from all of or a desired amount of the reactants of the reactant array 26. In one example, the detecting component 24 may be adjusted relative to the illumination component 12 and/or the light collection component 16 in response to actuation of the motor 20 such that different reactants are selectively positioned at a target area of the illumination component 12 and/or the light collection component 16. In one example, the motor 20 may be in communication with the adjustable stage 22 such that actuation of the motor 20 may cause the adjustable stage 22 to adjust and move (e.g., translate, rotate, etc.) the detecting component 24 relative to the illumination component 12 and/or the light collection component 16 (e.g., relative to the target area of the illumination component 12 and/or the light collection component 16), where the illumination component 12 and the light collection component 16 may be fixed relative to one another and/or other components of the fluid analysis system 10. Alternatively or additionally, one or both of the illumination component 12 and the light collection component 16 may be adjusted relative to the detecting component 24 in response to actuation of the motor 20.

The motor 20 may be any suitable type of device configured to couple with and adjust a position of the adjustable stage 22 and/or the detecting component 24 relative to the illumination component 12 and/or the light collection component 16. For example, the motor 20 may be a stepper motor, a continuous drive motor, a direct current (DC) motor, a servo motor, a manually operated handwheel, and/or other suitable device or system configured to produce motion. In some cases, the motor 20 may include a drive shaft configured to drive a driven component (e.g., the adjustable stage 22 and/or the detecting component 24 or other suitable driven component coupled with the adjustable stage 22 and/or the detecting component 24).

The motor 20 may be coupled with the adjustable stage 22 and/or the detecting component 24 in any suitable manner to facilitate a desired adjustment (e.g., linear adjustment, rotational adjustment, and/or other suitable adjustment) of the adjustable stage 22 and/or the detecting component in response to actuation of the motor 20. When the adjustable stage 22 and/or the detecting component 24 are to be adjusted in a linear manner, the coupling between the motor 20 and the adjustable stage 22 and/or the detecting component 24 may facilitate transferring the rotational motion of the motor 20 into linear motion of the adjustable stage 22 and/or the detecting component 24. When the adjustable stage 22 and/or the detecting component 24 are to be adjusted in a rotational manner, the coupling between the motor 20 and the adjustable stage 22 and/or the detecting component 24 may facilitate transferring rotational motion of the motor 20 into rotational motion of the adjustable stage 22 and/or the detecting component 24.

The motor 20 and the coupling with the adjustable stage 22 and/or the detecting component 24 may be configured to adjust a position of the adjustable stage 22 and/or the detecting component 24 at any suitable speed or rate. In some examples, the motor 20 may be configured to adjust the adjustable stage 22 and/or the detecting component 24 at a speed or rate in a range of less than 1 millimeter (mm)/second (s), in a range of about 1 mm/s to about 20 mm/s, in a range of 20 mm/s or greater, but other suitable ranges are contemplated. In some configurations, the motor 20 may be configured to continuously adjust the adjustable stage 22 and/or detecting component 24 at a constant speed or rate and/or change a speed or rate during a fluid test. In one example configuration, the motor 20 may be configured to adjust a position of the adjustable stage 22 and/or the detecting component 24 at a constant speed or rate of 5 mm/s during a fluid test.

The adjustable stage 22 may be any suitable component configured to support the detecting component 24 and/or the reactant array 26 (e.g., where the detecting component 24 may be a component having the surface 14). In some examples, the adjustable stage 22 may be or may include a platform coupled with the motor 20 (e.g., coupled directly or indirectly via a drive shaft of or extending from the motor 20) and configured to support one or more detecting components 24 including the reactant array 26 as the adjustable stage 22 is moved relative to the illumination component and/or the light collection component 16. Additionally or alternatively, the adjustable stage 22 may be or may include an arm coupled with the detecting component 24 and the motor 20 to transfer motion of the motor 20 to the detecting component 24. Further, in some examples, the adjustable stage 22 may be or may include the detecting component 24. For example, the adjustable stage 22 may be or may include the substrate 28 on which the reactant array 26 is located and as a result, may include the detecting component 24.

The substrate 28 of the detecting component 24 may have any suitable configuration for supporting and/or receiving the reactant array 26 for exposure to a fluid (e.g., a fluid of interest) and/or for analysis of the reactant array using the optical system of the system 10. For example, the substrate 28 may be sized to contain all of or a portion of the reactant array 26. In some examples, multiple substrates 28 may be utilized to contain all of or a portion of the reactant array 26. Additionally or alternatively, the substrate 28 and the reactant array 26 may be one in the same, such that reactant array 26 or reactants thereof form the substrate 28.

The substrate 28 may take on, or may have a surface (e.g., the surface 14) that may be, any suitable shape including, but not limited to, an elongated shape, a rectangular shape, a square shape, a rounded shape, a circular shape, a cylindrical shape, a disc shape, triangle shape, trapezoid shape, a prism shape, a lens shape, and/or other suitable shape. The substrate 28 may be or may include a surface of a container or cartridge or a component configured to be within a container or cartridge. In some instances, a cross-section of the substrate 28 may be symmetrical about a center line extending perpendicularly through a surface of the substrate configured to support one or more reactants of the reactant array 26.

The substrate 28 may include and/or may be formed from any suitable material. Example suitable materials used for the substrate 28 of the detecting component 24 include, but are not limited to, polymers, optical polymers, optical glasses, plastic, rubber, glass, paper, filter material, filter paper, fabric, metal, aluminum, polypropylene, polytetrafluorethylenes, porous membranes, chromatography plates, acrylic (e.g., poly (methyl methacrylate) (PMMA)), polycarbonate (PC), polystyrene (PS), other suitable materials, and/or combinations thereof. Further, the material utilized for the substrate 28 may be a solid material, a woven material, a hydrophobic material, a gas permeable material, a gas impermeable material, other suitable materials, and/or combinations thereof.

In one example configuration of the substrate 28, the substrate 28 may be or may include a portion that is formed from a porous white plastic membrane that has a high diffuse reflectivity over an entire visible spectrum, at least a portion of the ultraviolet (UV) spectrum, and/or at least a portion of the infrared (IR) spectrum. When the substrate 28 is at least partially formed from a white plastic membrane that has a high diffuse reflectivity over at least an entire visible spectrum, the light collection component 16 of the fluid analysis system 10 may be configured to collect a 100% white spectrum from the substrate 28, which may be used for fluid analysis purposes as discussed in greater detail herein.

In another example configuration of the substrate 28, the substrate 28 may be or may include a portion that is formed from a woven polypropylene material, which may result in a gas permeable, hydrophobic substrate 28. Although other pore sizes are contemplated, in the example configuration, the woven substrate may have an average pore size of or about 0.2 microns and a diameter of about 25 millimeters (mm). Additionally or alternatively, an example configuration of the substrate 28 may be formed from one or more other suitable hydrophobic, gas permeable materials.

In another example configuration of the substrate 28, the substrate 28 may be or may include a portion that is formed from a transparent material (e.g., acrylic (e.g., poly (methyl methacrylate) (PMMA)), polycarbonate (PC), polystyrene (PS), etc.) configured to pass light from one surface of the transparent material through a second surface of the material. In some examples, the substrate 28 may be entirely transparent or include one or more transparent portions configured to illuminate the reactants of the reactant array 26 through the substrate 28 and/or collect light from the reactants of the reactant array through the substrate 28. In some cases, the one or more transparent portions of the substrate 28 may extend between at least a first surface and a second surface of the substrate 28, where the first and second surfaces may be parallel or non-parallel with one another and the reactants are located on the first surface.

To increase fluid component detection rates by the reactants of the reactant array 26, the substrate 28 on which the reactant array 26 is applied and/or the reactants of the reactant array 26 may be textured (e.g., with grooves or surface topographical undulations, woven patterns, etc.) so as to increase an effective surface area of the reactants (e.g., the analyte sensitive material for detecting analytes). Additionally or alternatively, the reactants of the reactant array may be formed from a textured material and the substrate 28 may or may not be omitted. Such texturing may be applied to substrate 28 and/or the reactants of the reactant array 26 using any suitable technique including, but not limited to, via etching, thermoforming, pressure forming, molding, machining, weaving, three-dimensional printing, deposition, and/or other suitable techniques.

The reactants (e.g., analyte sensitive materials) of the reactant array 26 may be formed from any suitable material. In some cases, the material of the reactants may be an optically responsive chemical material (e.g., a chemoresponsive material) that changes color in response to detecting one or more analytes (e.g., non-volatile and/or volatile compounds, gases, liquids, and/or other fluids) in a fluid to which the reactants are exposed. Example suitable materials for reactants include dyes from, but not limited to, the following classes: Lewis acid/base dyes (e.g., metal containing dyes), Brensted acidic or basic dyes (e.g., pH indicators), dyes with large permanent dipoles (e.g., solvatochromic dyes), redox responsive dyes (e.g., metal nanoparticle precursors), and/or other suitable classes of dyes. One example material for the reactants may be a silver nanoparticle material. Other suitable materials for the reactants are contemplated, including reactant material that is not a printed dye.

In some examples, the material of the reactants may include an analyte sensitive material that is reversible or semi-reversible. Reversible or semi-reversible analyte sensitive material may be utilized for reactants configured for repeat monitoring, such as for continuous or periodic sensing of target locations to detect analytes from the target locations. Although other configurations of reactant arrays 26 are contemplated, example reactant arrays 26 including analyte sensitive material that is reversible or semi-reversible are discussed in U.S. Pat. No. 6,368,558 filed on Mar. 21, 2000, and titled COLORIMETRIC ARTIFICIAL NOSE HAVING AN ARRAY OF DYES AND METHOD FOR ARTIFICIAL OLFACTION; U.S. Pat. No. 6,495,102 filed on Nov. 11, 2000, and titled COLORIMETRIC ARTIFICIAL NOSE HAVING AN ARRAY OF DYES AND METHOD FOR ARTIFICIAL OLFACTION; U.S. Pat. No. 7,261,857 filed on Oct. 24, 2002, and titled COLORIMETRIC ARTIFICIAL NOSE HAVING AN ARRAY OF DYES AND METHOD FOR ARTIFICIAL OLFACTION; U.S. Pat. No. 8,852,504 filed on Oct. 11, 2007, and titled APPARATUS AND METHOD FOR DETECTING AND IDENTIFYING MICROORGANISMS, all of which are hereby incorporated by reference in their entirety and for all purposes.

In some examples, the material of the reactants may include an analyte sensitive material that is irreversible. Irreversible analyte sensitive material may be utilized for reactants configured for single use monitoring or single use monitoring per analyte material of a fluid when the reactant array 26 is configured to monitor for a plurality of different analytes, but this is not required. Although other configurations of reactant arrays 26 are contemplated, example reactant arrays 26 including analyte sensing material that is irreversible are discussed in U.S. Pat. No. 9,880,137 filed on Sep. 2, 2009, and titled COLORIMETRIC SENSOR ARRAYS BASED ON NANOPOROUS PIGMENTS; U.S. Pat. No. 10,539,508 filed on Jun. 9, 2015, and titled PORTABLE DEVICE FOR COLORIMETRIC OR FLUOROMETRIC ANALYSIS AND METHOD OF CONDUCTING COLORIMETRIC OR FLUOROMETRIC ANALYSIS; Li, Zheng, et al., “Ultrasensitive Monitoring of Museum Airborne Pollutants Using a Silver Nanoparticle Sensor Array”, ACS sensors 5.9 (2020): 2783-2791; Li, Zheng, and Kenneth S. Suslick, “Chemically Induced Sintering of Nanoparticles”, Angewandte Chemie 131.40 (2019): 14331-14334; LaGasse, Maria K., et al., “Colorimetric sensor arrays: Development and application to art conservation”, Journal of the American Institute for Conservation 57.3 (2018): 127-140, all of which are hereby incorporated by reference in their entirety and for all purposes.

The reactants of the reactant array 26 may be applied to the substrate 28 in any suitable manner. In one example, the reactants may be applied to the substrate 28 by printing the reactants (e.g., the material of the reactants) on the substrate 28. When printed, any suitable printing techniques may be utilized including, but not limited to, pin transfer, inkjet, silkscreen, and/or other suitable application techniques.

The reactants may be applied to the substrate 28 randomly and/or to form one or more patterns. Example configurations of the reactants of the reactant array 26 applied to the substrate 28 include, but are not limited to, grid patterns of rows and columns, concentric rings, color matching of a color of printed dye material with a color of a substrate material prior to interactions with analyte, patterns that result in identifiable shapes when the analyte sensitive material reacts to a particular analyte, other suitable configurations, and/or combinations thereof.

A top surface and/or other suitable surface of the substrate 28 may be coated with a porous material to increase the surface area when reactants are applied to the substrate 28. In one example, the top surface (e.g., the surface 14) of the substrate 28 may be coated with a thin layer of porous material, such as a sol-gel and/or other suitable material.

The fluid analysis system 10 may include an optics system configured to facilitate collecting photons to calculate a light collection measurement (e.g., reflectivity, photon count, intensity, etc.) of individual reactants of the reactant array 26. As discussed above, the optics system may include the illumination component 12 and/or the light collection component 16, among other suitable components.

In some examples, the optics system or a portion thereof may be configured to be stationary relative to the adjustable stage 22 and/or the detecting component 24. Alternatively or additionally, the optics system or a portion thereof may be configured to move or otherwise adjust relative to the adjustable stage 22 and/or the detecting component 24. In some configurations of the fluid analysis system 10, the adjustable stage 22 may be omitted and the detecting component 24 may be stationary as the optics system or a portion thereof is adjusted. Alternatively, the optics system or a portion thereof and the detecting component 24 may be stationary (e.g., fixed) relative to one another.

When included in the fluid analysis system 10, the illumination component 12 may include one or more light sources 30, an illumination lens system 32 (e.g., one or more illumination lens subsystems), and/or other suitable components. The illumination component 12 may be configured to provide sufficient photons with a uniform spatial and spectral distribution spanning a wavelength range of interest for the detecting component 24 to the reactants of the reactant array 26.

To maximize a signal to noise ratio for collecting light from the reactants of the reactant array 26 while minimizing consumed electrical power, an efficiency of electron to photon conversion of the one or more light sources 30 may be of interest. Also, efficiency in maximizing a ratio of collected photons to illumination photons may be considered. To facilitate maximizing the ratio of collected photons to illumination photons, the distribution of photons over the wavelength range of interest from the light sources 30 may be uniform. As such, utilizing an energy efficient light source that provides a uniform distribution of photons over the wavelength range of interest facilitates obtaining or calculating an accurate low noise light collection measurement (e.g., reflectivity, photon count, intensity, etc.) in every wavelength bin of the light collection component 16.

The one or more light sources 30 may be configured to provide any suitable wavelengths of light to one or more reactants. In some examples, the one or more light sources 30 may provide uniform spatial and spectral distributions of wavelengths of light spanning one or more ranges of, but not limited to, about 300 nanometers (nm) to about 1000 nm, a range of about 360 nm to about 900 nm, a range of about 350 nm to about 500 nm, a range of about 300 nm to about 600 nm, a range of about 400 nm to about 725 nm, a range of about 425 nm to about 725 nm, a range of 700 nm to about 1000 nm, a range of about 800 nm to about 1000 nm, and/or other suitable ranges of wavelengths of light. In one example, one or more light sources 30 may provide wavelengths of light spanning a range of about 400 nm to about 725 nm.

The optics system may be configured to provide illumination light in two or more different discrete ranges of wavelengths of light. For example, the one or more light sources 30 may provide light in a first range of wavelengths of light (e.g., about 300 nm to about 600 nm) and in a second range of wavelengths of light (e.g., about 800 nm to about 100 nm). The optics system may provide illumination in such two discrete ranges of wavelengths of light by utilizing two or more light sources 30, through the use of filters, and/or in one or more other suitable manners. Having the ability to provide light in two or more discrete wavelength ranges may facilitate using the fluid analysis system 10 for different applications that may require use of different wavelength ranges for optimal performance (e.g., optical detection of fluid components and/or other analytes).

In some configurations, the one or more light sources 30 may be configured to provide at least a uniform spatial and spectral distribution of broadband white light (e.g., continuous broadband white light) to one or more reactants of the reactant array 26. In one example, the light source 30 providing the uniform spatial and spectral distribution of broadband white light may provide light wavelengths spanning a range of about 360 nm to about 900 nm. In another example, the light source 30 providing the uniform spatial and spectral distribution of broadband white light may provide light wavelengths spanning a range of about 400 nm to about 725 nm. Such configured light sources 30 may have a desired (e.g., high) color rendering index (CRI), with a uniform distribution of photon wavelengths through the entire visible spectrum.

The one or more light sources 30 may be any suitable type of light source. For example, the light source 30 may be a light emitting diode (LED), an indium based blue LED with multiple phosphors added to a doping to create a combined LED and electro-luminescent semiconductor junction light emitting source, a black body radiation source, a tungsten lamp, a halogen lamp, and/or other suitable type of light source 30. In one example, the light source(s) 30 may be a true color white LED configured to provide light wavelengths in a range of about 400 nm to about 725 nm, but other suitable configurations are contemplated. Utilizing a white LED rather than a black body radiation source (e.g., tungsten lamps, halogen lamps, etc.) may reduce inefficiencies of electron to photon conversion and allow the fluid analysis system 10 to use less power (e.g., have a higher electron to photon conversion ratio) than when other types of light sources 30 (e.g., tungsten lamps, halogen lamps, etc.) are used.

The light sources 30 may be provided at any suitable angle and at any suitable location relative to the detecting component 24 (e.g., the reactant array 26 of the detecting component 24) and/or the light collection component 16. For example, the light sources 30 may be provided at angles in a range of about 0 degrees to about 90 degrees relative to the detecting component 24, at angles in a range of about 15 degrees to about 75 degrees relative to the detecting component 24, at angles in a range of about 30 degrees to about 60 degrees relative to the detecting component 24, at angles in a range of about 40 degrees and 50 degrees relative to the detecting component 24, and/or at one or more other suitable angles. In one example, the light sources 30 may be angled at 45 degrees relative to the detecting component 24, but other suitable configurations are contemplated. Providing light sources 30 that project light onto the reactants of the reactant array 26 from an acute angle and from a location spaced laterally from a target area (e.g., a lighted area) on the detecting component 24 may facilitate providing dual overlapping ellipsoids that effectively form the target area (e.g., form a target area sized to cover one or more reactants or portions of the one or more reactants) to be analyzed while minimizing collection of spectral or specular reflection light and allowing for maximum diffuse light collection.

The one or more light sources 30 may be configured in any suitable manner relative to the detecting component 24. In some examples and as discussed, the one or more light sources 30 may be configured relative to the detecting component 24 such that illumination may be projected on the detecting component 24 in a manner that prevents or mitigates spectral or specular reflections being captured by the light collection component 16 and maximizes capturing diffuse light from the detecting component 24 (e.g., reflections, etc. from the reactants of the reactant array 26). In one example, the one or more light sources 30 may include a first light source 30 and a second light source 30, where the first and second light sources 30 may be identical or different from one another and may be configured to illuminate a same target area on the detecting component 24. In some examples, the first and second light sources 30 and/or other light sources 30 may be positioned at a same angle relative to the detecting component 24 and at different locations relative to the detecting component 24, but other suitable configurations are contemplated.

In one example configuration of light sources 30 including the first and the second light sources 30, the first light source 30 may be at a first location and a first angle relative to the detecting component 24 and the second light source 30 may be at a second angle and a second location relative to the detecting component 24. The first angle and the second angle may be a same angle or a different angle. In one example, the first angle and the second angle may be a same angle and may be about 45 degrees relative to the detecting component 24 (e.g., relative to the reactant array 26 or surface supporting the reactant array 26). The first location and the second location may be different locations and in one example, the first location and the second location may oppose one another such that light from a same angle, but opposite directions, is applied to the detecting component 24 to form a target area on the detecting component 24. Other suitable configurations are contemplated.

The one or more light sources 30 may be configured to applying light to the reactants of the reactant array directly and/or indirectly through the substrate 28 of the detecting component 24. For example, one or more light sources 30 may directly illuminate the reactants of the reactant array 26 from a location above the substrate 28 and/or indirectly illuminate the reactants from a location below and through the substrate 28 (e.g., through an at least partially transparent portion of the substrate 28).

In some instances, direct illumination of reactants with one or more light sources 30 may cause or increase the potential for causing an interference with collecting light from the reactants and limit light collection efficiency. To facilitate resolving this light collection efficiency issue, the light sources 30 may illuminate the reactants through the substrate 28 and/or at a side of the reactants of the reactant array opposing a side at which light from the reactants is collected, which may facilitate avoiding interference between illumination optics and/or mechanics with diffused light collection optics that can limit an overall light collection efficiency of the system. Such improvements, along with other improvements, to light collection efficiency may reduce energy consumption and drain on a battery such that the fluid analysis system 10 may last longer on a charge of a battery.

The illumination lens system 32, when included, may be configured to deliver and focus light from the light source 30 on or to create a target area on the detecting component 24. In some examples, the target area on the detecting component 24 may cover or include one or more reactants of the reactant array 26, but other suitable target areas are contemplated. The illumination lens system 32 may include any suitable components including, but not limited to, one or more lenses, one or more mirrors, one or more fiber optics, and/or one or more other suitable components.

When one or more fiber optics (e.g., one or more optical fibers) are included in the illumination lens system 32, the fiber optics may be configured (e.g., tuned and positioned) to deliver light to or focus light on one or more reactants of the reactant array 26 from the light source(s) 30. The fiber optics may be single mode and/or multimode fiber optics, as desired.

The one or more lenses, when included in the illumination lens system 32, may be configured (e.g., tuned and positioned) to deliver light to or focus light on the target area (e.g., one or more reactants of the reactant array 26) from the light source(s) 30. The one or more lenses may include a configuration with a single lens or a configuration with two or more lenses that may be similar or different than one another. In some examples, the illumination lens configuration may include a first lens and a second lens that may operate together to deliver light to or focus light on the target area. In one example, the first lens may be located between the light source 30 and the second lens and may be a convex lens (e.g., an aspheric lens) and the second lens may be located between the first lens and the target area on the detecting component 24 and may be a cylinder lens or other suitable lens configured to focus light from the light source 30 onto the target area at the detecting component 24.

In some examples, the one or more lenses of the illumination lens system 32 may have a diameter and a focal length, where a ratio of the diameter to the focal length (e.g., a lens F number) may be in a range of about 0.5 to about 2.0, in a range of about 0.75 to about 1.5, in a range of about 0.8 to about 1.2, in a range of about 0.9 to about 1.1, and/or within one or more other suitable range. In one example, the one or more lenses of the illumination lens system 32 may have a diameter to focal length of about 1.0. Further, although not required, the one or more lenses may include a short focal length convex lens that may be configured to match a spot size on the detecting component 24 from a light source 30 with a size of a target area (e.g., one or more reactants) on the detecting component 24.

The light collection component 16 (e.g., diffuse reflection capture optics, etc.) may be configured to collect and measure levels of or changes in wavelengths of light collected from the surface 14 (e.g., measure photons by wavelengths of light from individual reactants of the reactant array 26) and may include one or more light collectors 34, a collection lens system 36 (e.g., a collection lens subsystem), and/or other suitable components. The light collection component 16 may be configured to be focused on the target area (e.g., an illuminated portion of the detecting component 24, which may include a reactant of the reactant array 26) to avoid or mitigate collecting light from spaces (e.g., white spaces) between reactants and/or from more than one reactant. Further, focusing the light collection component 16 on a single reactant may facilitate obtaining light from an entirety of or at least a majority of the single reactant, which may minimize the likelihood of obtaining skewed light measurements from the reactant due to printing defects, granularity in the material used for the reactants, defects in the substrate 28, and/or due to other irregularities. In some examples, at least a portion of the light collection component 16 (e.g., a portion of the light collection component 16 proximate the detecting component 24) may be oriented perpendicular to or substantially perpendicular to substrate 28 and/or a surface of the detecting component 24 to minimize collecting or receiving light from spectral or specular reflections and maximize light collection from the target area.

The light collection component 16 may be positioned at any suitable location relative to the detecting component 24. In some examples, the light collection component 16 may be configured to collect light from a same side of the detecting component 24 from which the illumination component 12 illuminates the detecting component 24, from a different side of the detecting component 24 than from which the illumination component 12 illuminates the detecting component 24, directly from the reactant of the reactant array 26, indirectly through a transparent substrate 28 of the detecting component 24, and/or from one or more other suitable location and/or in one or more other suitable manners.

The collection lens system 36, when included, may be configured to receive or collect light from the target area at the detecting component 24 and focus an aperture on the target area (e.g., the focus of the aperture may be slightly smaller than the illumination spot from the illumination component 12) or focus light from the target area at the detecting component 24 for collection by the light collector 34. The collection lens system 36 may include any suitable components including, but not limited to, one or more lenses, one or more mirrors, one or more fiber optics, and/or one or more other suitable components.

When one or more fiber optics (e.g., one or more optical fibers) are included in the collection lens system 36, the fiber optics may be configured (e.g., tuned and positioned) to receive light from or focus light from one or more reactants of the reactant array 26. The one or more fiber optics may be or may include single mode and/or multimode fiber optics, as desired. The one or more fiber optics may have a first end configured to receive or collect light from the target area and a second end in optical communication with the light collector 34.

The one or more lenses, when included in the collection lens system 36, may be configured (e.g., tuned and positioned) to receive, collect, and/or focus light from the target area (e.g., from one or more reactants of the reactant array 26) and direct the light to the light collector 34 (e.g., an image sensor or wave guide in optical communication with a sensor or other component of the light collector). The one or more lenses may include a configuration with a single lens or a configuration with two or more lenses that may be similar or different than one another. In some examples, the collection lens system 36 may include a first lens and a second lens that may operate together to obtain light from the target area (e.g., light from substantially only the target area) and deliver light to or focus light on a sensor of the light collector 34.

In one example configuration of the collection lens system 36 with two lenses and a fiber optic wave guide in communication with the light collector 34, the first lens may be located between the light collector 34 or a wave guide (e.g., a fiber optic) of the light collector 34 in optical communication with a sensor of the light collector 34 and may be a convex lens (e.g., an aspheric lens) configured to focus light from the target area at the detecting component 24 (e.g., via the second lens) on the sensor and/or an inner core of the wave guide. The second lens may be located between the first lens and the target area at the detecting component 24 and may be a cylinder lens or other suitable lens configured to collect light from an entirety of or substantially an entirety of the target area at the detecting component 24 (e.g., collect light from a reactant of the reactant array 26).

A focal length of the combination of first lens and the second lens, a distance from the second lens to the target area, and a distance to an inner core of the fiber optic wave guide from the first lens may be chosen or selected to give precise magnification and dimensions required for an acceptance aperture configured to optimize overall illuminator to light collector photon utilization efficiency, minimize electrical power required to sense light from the detecting component 24, and ensure the light collector integration time may be minimized. In turn, this may allow a sample rate of the light collector 34 to be increased, which may reduce an amount of time needed to capture all of the individual spectra required to fully characterize the response of every reactant or at least a desired subset of reactants in the reactant array 26 for use in analyzing a fluid sensed by the reactant array 26.

The one or more light collectors 34 of the fluid analysis system 10 may be any suitable type of light collector. Example suitable types of light collectors 34 may include, but are not limited to, an image sensor, an n-dimensional sensory array (e.g., where “n” equals 1, 2, etc.), a spectrometer, a charge-coupled device (CCD) image sensor, complementary metal-oxide semiconductor (CMOS) image sensor, contact image sensor (CIS), color contact image sensor (CCIS), a camera, other suitable light collectors, and/or combinations of light collectors. In one example, the light collection component 16 may include a spectrometer configured to measure photons collected from (e.g., reflected, transmitted, and/or otherwise received from) the target area. Utilizing the spectrometer may facilitate sensing wavelengths of light with high resolution in the nanometer range and may provide a continuous set of data over the wavelength range, which allows for a sensitive analysis of the data to identify components of a fluid to which the reactant array 26 was exposed relative to when other light collectors are used.

When a spectrometer is utilized as the light collector 34, any suitable type of light collector 34 configured to measure (e.g., measure over time) levels of wavelengths of light collected from the surface 14 (e.g., from the detecting component 24) may be utilized. In some examples, the spectrometer may have a compact folded optical system with a diffraction grating and a linear imager (e.g., a linear array photo detector, a CCD linear imager, and/or other suitable type of linear imager), where the diffraction grating is configured to output specific wavelengths of light received or collected at specific, consistent locations (e.g., pixels forming bins or groups of wavelengths) on the linear imager. In some cases, the diffraction grating of the spectrometer may be configured to divide receive or collected light into bins or groups spanning 1 nm or less, where the smaller the bin the greater the resolution of the data related to the reactants of the reactant array 26. In one example, the spectrometer may be configured to sense wavelengths over a range of about 390 nm to about 950 nm and divide light from the spectrum into 1 nm bins. Other suitable configurations of spectrometers are contemplated.

The controller 18 may be coupled to one or more other electronic components of the fluid analysis system 10. For example, the controller 18 may be communicatively coupled with one or more of the illumination component 12, the light collection component 16 (e.g., the light collector 34 and/or other components of the light collection component 16), the motor 20, and/or one or more other suitable components of the fluid analysis system 10 and/or remote components (e.g., servers, mobile devices, etc.) that may or may not be part of the fluid analysis system 10. In some examples, the controller 18 may be configured to receive an indication to initiate a fluid analysis test (e.g., from a user via a user interface or in communication with the controller 18) and send coordinated control signals to the motor 20, the one or more light sources 30, and the light collector 34 to initiate movement of the motor 20 to adjust a location of the detecting component 24 relative to the illumination component 12 and the collection lens system 36, to initiate illumination of a target area on the detecting component 24, and initiate sensing wavelengths of light from the reactant array 26 or other suitable target area of the detecting component 24.

The controller 18 may be configured to identify or may facilitate identifying a component of fluid in contact with the detecting component 24 (e.g., including the surface 14) based on measured (e.g., sensed and/or calculated) levels of or changes in wavelengths of light collected from the detecting component 24 with the light collection component 16 (e.g., via the spectrometer and/or other suitable light collector 34). In some examples, the controller 18 may be configured to identify the component of fluid in contact with the detecting component 24 based on one or both of a timing of the levels of the wavelength of light reflected off of the detecting component 24 and an absolute change between a level of a wavelength of light collected from the surface at a time of or prior to an application of the fluid to the detecting component 24 and at a predetermined time after initially applying the fluid to the detecting component 24. The controller 18 may be configured to identify the component of the fluid in contact with the detecting component 24 in one or more additional or alternative manners.

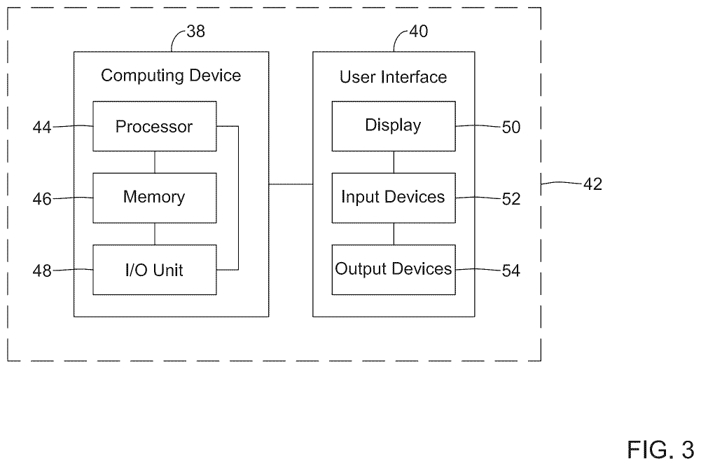

The controller 18 and/or other components of the fluid analysis system 10 may be or may include one or more computing devices including or coupled with one or more user interfaces. FIG. 3 depicts a schematic diagram of an illustrative computing device 38 and a user interface 40, where the computing device 38 and/or the user interface 40 may be entirely or partially housed in one or more housings 42 (e.g., a housing which may or may not house other components of the fluid analysis system 10). The housing 42 may be an optional component, as represented by the broken lines defining the housing 42 depicted in FIG. 3. Although various components are depicted as being included in the computing device 38 and the user interface 40, one more of the depicted components may be omitted and/or one or more additional or alternative components may be utilized.

The computing device 38 may be any suitable computing device configured to process data of or for the fluid analysis system 10 and may be configured to facilitate operation of the fluid analysis system 10. The computing device 38, in some cases, may be configured to control operation of the fluid analysis system 10 by establishing and/or outputting control signals to the illumination component 12, the light collection component 16, the motor 20, and/or other electronic components of the fluid analysis system 10 to run a fluid analysis test and/or monitor results of a fluid analysis test. In some examples, the computing device 38 may be part of the controller 18 and may communicate with other components over a wired or wireless connection, but other suitable configurations are contemplated. When the computing device 38, or at least a part of the computing device 38, is a component separate from a structure of the controller 18, the computing device 38 may communicate with electronic components of the fluid analysis system 10 over one or more wired or wireless connections or networks (e.g., LANs and/or WANs). In some cases, the computing device 38 may communicate with a remote server or other suitable computing device.

The illustrative computing device 38 may include, among other suitable components, one or more processors 44, memory 46, and/or one or more I/O units 48. Example other suitable components of the computing device 38 that are not specifically depicted in FIG. 3 may include, but are not limited to, communication components, a touch screen, selectable buttons, and/or other suitable components of a controller. As discussed, one or more components of the computing device 38 may be separate from the controller 18 and/or incorporated into the components of the controller 18.

The processor 44 of the computing device 38 may include a single processor or more than one processor working individually or with one another. The processor 44 may be configured to receive and execute instructions, including instructions that may be loaded into the memory 46 and/or other suitable memory. Example components of the processor 44 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 46 of the computing device 38 may include a single memory component or more than one memory component each working individually or with one another. Example types of memory 46 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 46 may be or may include a non-transitory computer readable medium. The memory 46 may include instructions stored in a transitory state and/or a non-transitory state on a computer readable medium that may be executable by the processor 44 to cause the processor 44 to perform one or more of the methods and/or techniques described herein. Further, in some cases, the memory 46 and/or other suitable memory may store data received from the light collector 34, the motor 20, the light sources 30, and/or other components of or in communication with the fluid analysis system 10.

The I/O units 48 of the computing device 38 may include a single I/O component or more than one I/O component each working individually or with one another. Example I/O units 48 may be or may include any suitable types of communication hardware and/or software including, but not limited to, communication components or ports configured to communicate with electronic components of the fluid analysis system 10 and/or with other suitable computing devices or systems. Example types of I/O units 48 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOH protocol components, Near-Field Communication (NFC) protocol components, WI-FI protocol components, optical communication components, ZIGBEE protocol components, and/or other suitable wireless communication components), and/or other suitable I/O units 48.

The user interface 40 may be configured to communicate with the computing device 38 via one or more wired or wireless connections. The user interface 40 may include one or more display devices 50, one or more input devices 52, one or more output devices 54, and/or one or more other suitable features. In some examples, the user interface 40 may be part of or may include the computing device 38.

The display 50 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 52 may be and/or may include any suitable components and/or features for receiving user input via the user interface 40. Example input device(s) 52 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 52 may include a touch screen that allows for setting set points, initiating a fluid analysis test, adjusting between screens (e.g., a testing screen, a data analysis screen, a results screen, etc.) and/or allows for taking one or more other suitable actions.

The output device(s) 54 may be and/or may include any suitable components and/or features for providing information and/or data to users and/or other computing components. Example output device(s) 54 include, but are not limited to, displays, speakers, vibration systems, tactile feedback systems, optical outputs, and/or other suitable output devices.

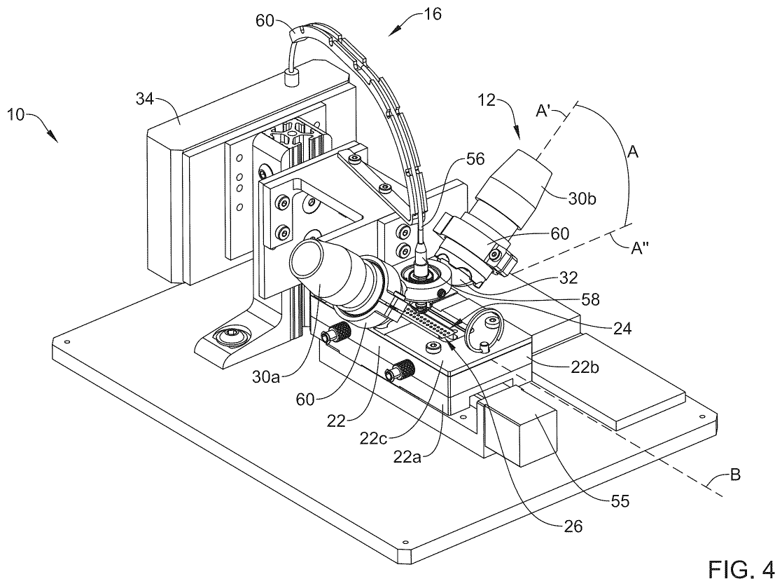

FIG. 4 depicts a schematic perspective view of an illustrative configuration of the fluid analysis system 10, where the fluid analysis system 10 may be configured to receive a detecting component 24 with a linear reactant array 26. The illustrative configuration of the fluid analysis system 10 depicted in FIG. 4 may further include the illumination component 12, the light collection component 16, the adjustable stage 22, and/or other suitable components. Further, components of the fluid analysis system 10 depicted in FIG. 4 and not discussed here may optionally be omitted and/or other components not depicted in FIG. 4 may be included, as desired. In one example, although the controller 18 is not depicted in FIG. 4 as being part of the fluid analysis system 10, the fluid analysis system 10 may include or may be configured to couple with and/or otherwise communicate with the controller as discussed herein and/or in other suitable manners.

The illustrative configuration of the fluid analysis system 10 may include the motor 20 (e.g., not shown in FIG. 4, but represented by a motor housing 55 in FIG. 4) configured to drive or otherwise translate the adjustable stage 22 in opposing directions along axis B. When the reactant array 26 of the detecting component 24 includes a plurality of rows and columns, the motor 20 and associated gearing may be configured to adjust the adjustable stage 22, and thus the detecting component 24, in one or more direction transverse to the axis B (e.g., a direction perpendicular to the axis B and/or other suitable directions).

The illustrative configuration of the fluid analysis system 10 may or may not include a housing and may be configured to be a handheld fluid analysis system 10 and battery powered. Alternatively, the illustrative configuration of the fluid analysis system 10 may or may not include a housing and may be configured to be a bench top fluid analysis system 10.

The adjustable stage 22 may take on any suitable configuration configured to support the detecting component 24 and may have one or more components. In some examples, the adjustable stage 22 may have a first component 22a, which may be a base that is configured to engage a gear or be driven in one more suitable manners, a second component 22b, which may be a body that is configured to receive the detecting component 24, and a third component 22c, which may be a cover that is configured to facilitate maintaining a position of the detecting component 24 at or within the adjustable stage 22. Alternatively, the detecting component 24 may include the first component 22a, the second component 22b, the third component 22c, and/or other components of the adjustable stage 22. One or more of the first component 22a, the second component 22b, and the third component 22c may be sub-components or portions of a single component or may be components that may be engaged with one another to form the adjustable stage 22 When two or more of the first component 22a, the second component 22b, and the third component 22c are components engageable together to form at least part of the adjustable stage 22 and/or the detecting component 24, two or more of the first component 22a, the second component 22b, and the third component 22c may be coupled together in one or more suitable manners. For example, the first component 22a, the second component 22b, and the third component 22c may be coupled together using one or more threaded components (e.g., screws, etc.), one or more pins, one or more snap connections, one or more friction connections, one or more adhesives, one or more welds, and/or using one or more other suitable coupling techniques. In some cases, a coupling technique for coupling the second component 22b with the third component 22c may be reversible to facilitate separating the second component 22b and the third component 22c for inserting and/or removing the detecting component 24 from the adjustable stage 22. Other suitable configurations of the adjustable stage 22 are contemplated.

The components of the adjustable stage 22 may be configured to receive and align the detecting component 24 relative to the illumination component 12 and/or the light collection component 16. In some examples, the components of the adjustable stage 22 may include a recess configured to receive the detecting component 24

The illumination component 12 of the illustrative configuration of the fluid analysis system 10 depicted in FIG. 4 may include a first light source 30a and a second light source 30b. As depicted in FIG. 4, the first light source 30a and the second light source 30b may be above and at an angle A with a surface of the detecting component 24 supporting the reactant array 26 (e.g., a surface of the substrate 28 and/or other suitable surfaces). Angle A may extend between line A′ representing an axis through an individual light source 30 and line A″ representing a line that is parallel to the surface of the detecting component 24 supporting the reactants of the reactant array 26. The angle A may be any suitable acute angle, such as 45 degrees or other suitable angle.

As discussed, the light sources 30 may be at any suitable location relative to one another and relative to the detecting component 24 that is configured to illuminate a target area at the detecting component 24. When the light sources 30 are configured to be stationary or at a fixed location relative to an entirety of or at least a portion of the light collection component 16, the target area at the detecting component 24 may be whichever portion of detecting component 24 (e.g., a location of one or more reactants of the reactant array 26) that has been translated to a location illuminated by the light sources 30. As depicted in FIG. 4, the first light source 30a and the second light source 30b may oppose one another such that first light source 30a is spaced 90 degrees from the second light source 30b, but other suitable configurations are contemplated.

Each of the first light source 30a and the second light source 30b may include the illumination lens system 32 (only the second light source 30b is depicted in FIG. 4 with the illumination lens system 32 due to an angle of the view of the fluid analysis system 10). The illumination lens system 32 may be configured to focus illumination from the respective light source 30 to the target area at the detecting component 24 (e.g., on one or more reactants of the reactant array 26, etc.)

The light collection component 16 of the configuration of the fluid analysis system 10 depicted in FIG. 4 may further include the light collector 34 (e.g., a spectrometer and/or other suitable light collector 34), an optical fiber 56 and a lens housing 58 configured to house the collection lens system 36 (not depicted in FIG. 4) that includes one or more lenses configured to facilitate collecting light from the detecting component 24. In some configurations, the optical fiber 56 may be configured to extend from the lens housing 58 to the light collector 34 and guide light collected at the collection lens system 36 (e.g., from the detecting component 24 and/or other suitable surface 14) to the light collector 34. The optical fiber 56 may be utilized or omitted based at least in part on a type of light collector used (e.g., the optical fiber 56 may facilitate collecting light when collecting light with a spectrometer, but may, optionally, be omitted when collecting light with a contact image sensor or camera).

The fluid analysis system 10 depicted in FIG. 4 may include one or more supports 60. For example, the one or more supports 60 may include one or more supports 60 configured to support the light source 30 (e.g., the first light source 30a and the second light source 30b), support the optical fiber 56 between the light collector 34 and the collection lens housing 58, support the collection lens housing 58 relative to the detecting component 24, and/or support one or more additional and/or alternative components of the fluid analysis system 10 relative to other components thereof.

As discussed above, when the illumination component 12 of the fluid analysis system 10 is configured to directly illuminate a detecting component 24 from above the detecting component 24 and at a same side of the detecting component 24 from which the light collection component 16 collects light from reactants of the detecting component 24, the light collection component 16 may inadvertently collect light from the light sources 30 that is from the detecting component 24, but that is not from the reactant on the detecting component 24. To reduce light collected from sources other than reactants of the detecting component 24, the illumination component 12 may be configured to apply light to the reactants through the substrate 28 of the detecting component 24.

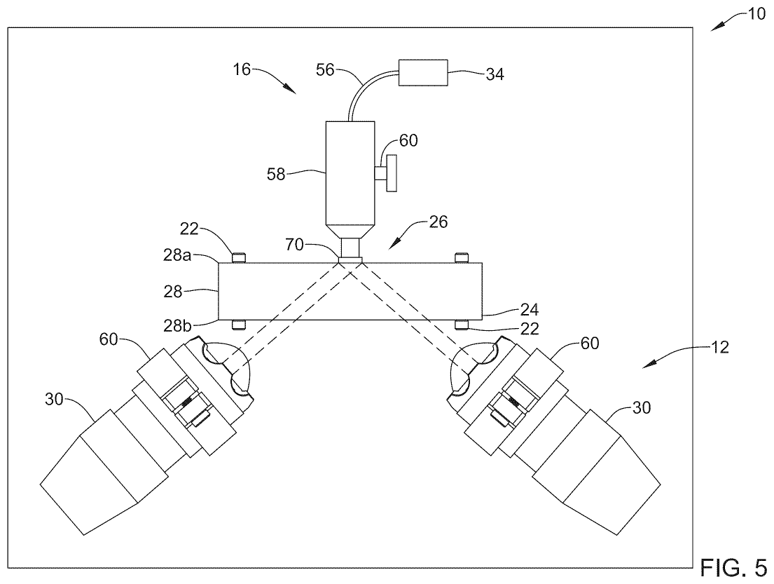

FIG. 5 depicts a schematic side view of an illustrative configuration of the fluid analysis system 10, where the illumination component 12 may illuminate a reactant 70 on the detecting component 24 through the substrate 28 and the light collection component 16 may collect light from the reactant 70 of the reactant array 26 without collecting the light directly passing through the substrate 28. To apply light to the reactant(s) 70 of the reactant array 26 through the substrate 28, the substrate 28 may be or may include transparent portions extending between and/or configured to allow light to travel from a second surface 28b of the substrate 28 to a first surface 28a of the substrate 28 at which the reactant(s) 70 may be located. The broken lines in FIG. 5 schematically depict light paths from the light sources 30, through transparent portions of the substrate 28, to the reactant 70. The solid lines from the reactant 70 to the light collection component 16 schematically depict diffusely reflected or scattered or reemitted light from the reactant(s) that is collected by the light collection system. Although not required, the configuration of the fluid analysis system 10 depicted in FIG. 5 may be configured similar to and/or function similar to the configuration of the fluid analysis system 10 depicted in FIG. 4, other than utilizing a transparent substrate 28 through which the light sources 30 illuminate the reactant(s) 70.

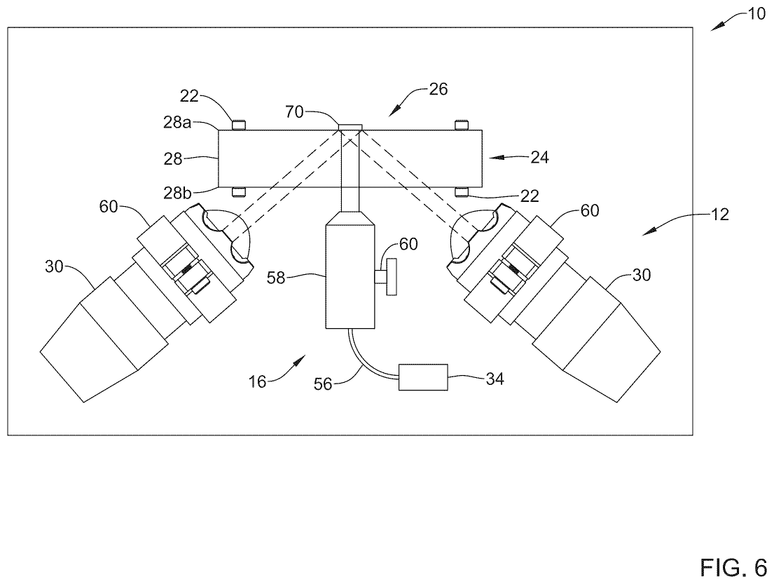

FIG. 6 depicts a schematic side view of an illustrative configuration of the fluid analysis system 10, where the illumination component 12 may illuminate a reactant(s) 70 on the detecting component 24 through the substrate 28 and the light collection component 16 may collect light from the reactant(s) 70 that has traveled through the substrate 28. In some configurations and as depicted in FIG. 6, the light collection component 16 may collect light from the reactant(s) 70 that is received through a surface of the substrate 28 that opposes a surface of the substrate 28 supporting the reactant(s) 70, but other suitable configurations are contemplated. To apply light to the reactant(s) 70 of the reactant array 26 through the substrate 28 and collect light from the reactant(s) 70 through the substrate 28, the substrate 28 may be or may include transparent portions extending between and/or configured to allow light to travel to and/or from a second surface 28b of the substrate 28 to a first surface 28a of the substrate 28 at which the reactant(s) 70 is located. The broken lines in FIG. 6 schematically depict light paths from the light sources 30, through transparent portions of the substrate 28, to the reactant(s) 70. The solid lines from the reactant(s) 70, through transparent portions of the substrate 28, to the light collection component 16 schematically depict diffusely reflected or scattered or reemitted light from the reactant(s) that is collected by the light collection system. Although not required, the configuration of the fluid analysis system 10 depicted in FIG. 6 may be configured similar to and/or function similar to the configuration of the fluid analysis system 10 depicted in FIG. 4, other than illuminating the reactant(s) 70 through the transparent substrate 28 and collecting light from the reactant(s) 70 of the reactant array 26 through the transparent substrate 28.

Applying light from the light source(s) 30 to the reactants 70 of the reactant array 26 through the transparent substrate 28 and collecting light from the reactants 70 through the transparent substrate 28 may facilitate positioning the reactants 70 close to a source of fluid to be analyzed by the fluid analysis system 10 due to the illumination component 12 and the light collection component 16 being positioned proximate an opposing side(s) of the substrate 28 relative to a side at which the reactants 70 are located. In one example, the reactants 70 may be positioned adjacent to a wound from which analytes (e.g., volatile organic compounds (VOCs) and/or other analytes) are emitted and light from the reactants 70 may be collected and analyzed in real-time as the reactants 70 are exposed to the VOCs from the wound. Other suitable examples are contemplated.

Applying light from the light source 30 to the reactant(s) 70 of the reactant array 26 through the transparent substrate 28 (e.g., as depicted in FIGS. 5, 6, 7 and 8) may facilitate illuminating or exciting the reactant(s) 70 in any suitable manner. For example, applying the light through the transparent substrate 28 to the reactant(s) 70 may illuminate and/or excite the reactant(s) through evanescent illumination light, direct large glancing angle light, a combination of evanescent illumination light and direct large glancing angle light, and/or other suitable light techniques.

Applying light through the transparent substrate 28 to the reactant(s) 70 in the manner discussed may ensure light from the light source(s) 30 is directed to the target area at which the reactant(s) 70 is located (e.g., via an evanescent light wave, etc.) and directly away from the light collection component 16, such that no or minimal light from the light sources 30 is directly collected by the light collection component 16 in addition to the diffused light collected from the reactant(s) 70. As such, illuminating the reactant(s) 70 through the transparent substrate 28, which directs light from lights sources 30 away from the light collection component 16, may facilitate reducing a size of the fluid analysis system 10 by allowing the light collection component 16 to be positioned closer to the reactant(s) 70 from the reactant surface side than in other configurations that are configured to illuminate the reactant(s) from the same side as the light collection component 16 and thus, require the light collection component 16 to be positioned relatively far from the reactant(s) 70 to mitigate mechanical interference. It should be noted that the same is also true if the illumination components are positioned on the reactants surface side and the light collection components are positioned on the other side of the transparent substrate also such a configuration is not schematically shown here.

The transparent substrate 28 may have any suitable shape or size, as discussed herein. In some examples, a shape of the transparent substrate 28 may have a lensing or prism effect on light passing through the transparent substrate 28 (e.g., the transparent substrate 28 may act as a light guide or a light beam shaper). In some examples, a lensing effect on light passing through the transparent substrate 28 may be achieved by configuring one or more refractive indices of the substrate 28 or surface profiles. The refractive index of the transparent substrate 28 may be tuned or configured in any suitable manner including, but not limited to, by using plasmonic metasurfaces, dielectric metasurfaces, and/or other suitable configurations or techniques. In some examples, the transparent substrate bottom or side surfaces may be made to have a curved surface profile in one or two dimensions to serve the function of light beam guiding and/or shaping.

When the transparent substrate 28 is configured to have a lensing or prism effect on light passing therethrough, an angle at which the light impinges on a surface of the substrate 28 supporting the reactant(s) 70 may dictate a direction of travel of the light. For example, when light is directed through the transparent substrate 28 to a surface on which the reactant(s) 70 is located at an angle that is less than a critical angle (e.g., where a critical angle may be about 45 degrees with respect to the surface of the substrate 28 at which the reactant(s) 70 is located), the light may be refracted and exit or leave the surface at which the reactant(s) 70 are located at a large glancing angle. When light is directed through the transparent substrate 28 to a surface on which the reactant(s) 70 is located at an angle that is greater than the critical angle, the light may be totally internally reflected such that the light leaves the transparent substrate 28 through a surface other than the surface at which the reactant(s) 70 is located and such that an evanescence wave is created that may penetrate beyond the surface of the substrate 28 at which the reactant(s) 70 is located and to the reactant(s) 70. In some cases, an amplitude of the created evanescence wave may decrease exponentially from the surface at which the reactant(s) 70 are located.

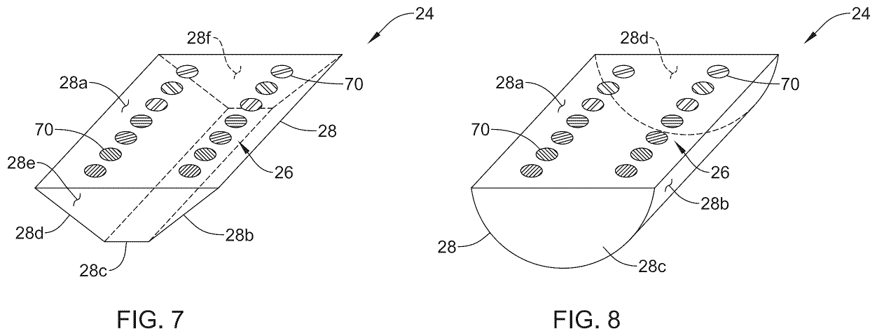

FIG. 7 schematically depicts a perspective view of an illustrative configuration of the detecting component 24 (e.g., a CSA) including a transparent substrate 28 having a trapezoid cross-section shape that may facilitate directing light away from the light collection component 16. As depicted in FIG. 7, the reactants 70 of the reactant array 26 may be on or may be supported by a first surface 28a of the substrate 28. The transparent substrate 28 may include a second surface 28b, a third surface 28c, and a fourth surface 28d, which may form the trapezoid cross-section shape with the first surface 28a. A fifth surface 28e and a sixth surface 28f of the transparent substrate 28 may form opposite ends of the substrate 28. In some examples, the second surface 28b, the fourth surface 28d, the fifth surface 28e, and the sixth surface 28f in the configuration of the transparent substrate 28 depicted in FIG. 7 may be non-parallel with the first surface 28a and the third surface 28c may be parallel with the first surface 28a. In some examples, the trapezoid cross-section shape of the substrate 28 may be symmetrical about a center line extending perpendicularly through the first surface 28a, but other suitable configurations are contemplated.

FIG. 8 schematically depicts a perspective view of an illustrative configuration of the detecting component 24 (e.g., a CSA) including a transparent substrate 28 having a semi-circular cross-section shape. As depicted in FIG. 8, the reactants 70 of the reactant array 26 may be on or may be supported by a first surface 28a of the substrate 28. The transparent substrate 28 may include a second surface 28b (e.g., a rounded surface), which may form the semi-circular cross-section shape with the first surface 28a. A third surface 28c and a fourth surface 28d of the transparent substrate 28 may form opposite ends of the substrate 28. In some examples, the second surface 28b, the third surface 28c, and the fourth surface 28d in the configuration of the transparent substrate 28 depicted in FIG. 8 may be non-parallel with the first surface 28a. Although the second surface 28b of the transparent substrate 28 in FIG. 8 may have a line along its length that has a tangential plane which is parallel to the first surface 28a, the second surface 28b may be considered to be non-parallel with the first surface 28a due to the continuous curve of the second surface 28b. In some examples, the semi-circular cross-section shape of the substrate 28 may be symmetrical about a center line extending perpendicularly through the first surface 28a, but other suitable configurations are contemplated.

Light applied through the transparent substrate 28 (e.g., the transparent substrates in FIGS. 7 and 8 and/or other suitable transparent substrates) may be applied to any suitable side of the substrate 28 that facilitates directing light from the light source(s) 30 to the reactants 70 of the reactant array 26. In one example, the reactant array 26 may be on or may be supported by a first surface of the transparent substrate 28 and the light source 30 may be applied to a second surface of the transparent substrate, which may be parallel or non-parallel with the first surface (e.g., as depicted in FIGS. 5 and 6). In another example, the reactant array 26 may be on or supported by a first surface of the transparent substrate 28 and a first light source may apply light to the reactants 70 of the reactant array 26 through a second surface of the substrate 28 and a second light source may apply light to the reactants 70 through a third surface of the substrate, where the second surface and the third surface may be parallel or non-parallel with respect to each other and/or with respect to the first surface.

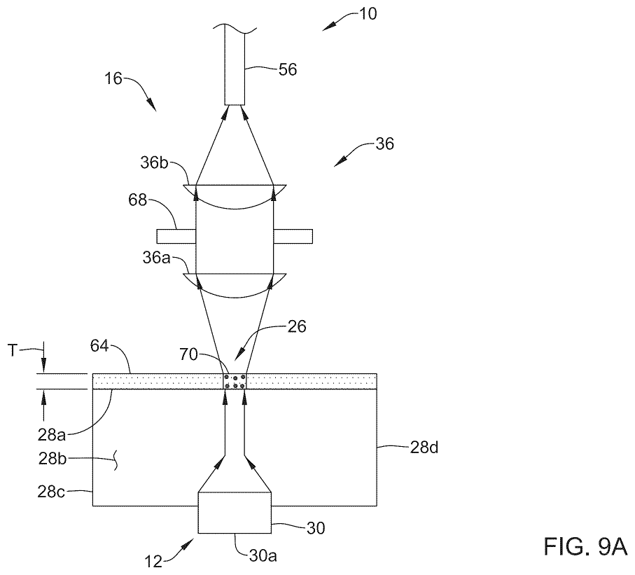

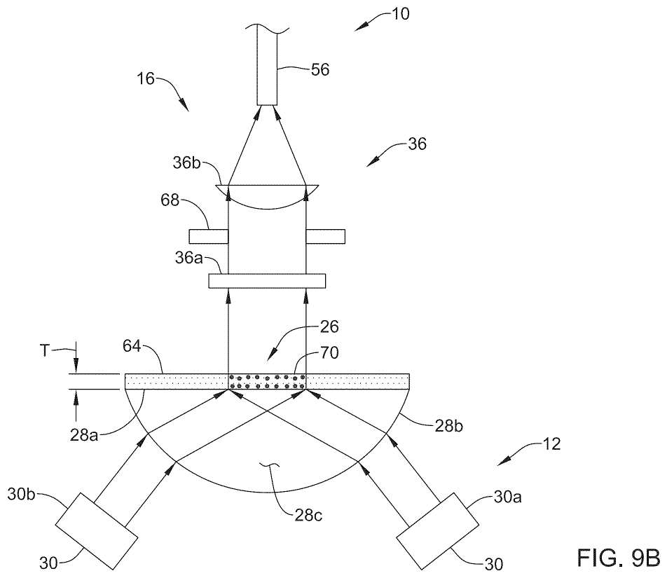

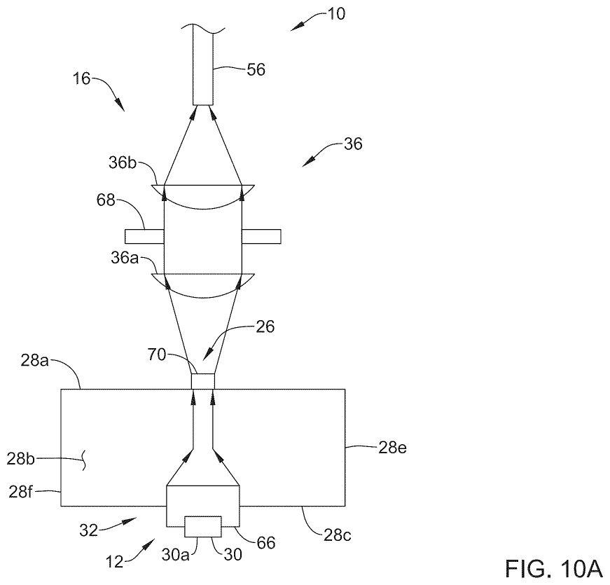

FIGS. 9A and 9B depict schematic side and end views, respectively, of a portion of the fluid analysis system 10 including the illumination component 12, the light collection component 16, and a transparent substrate 28 having a semi-circular cross-section shape having first through fourth surfaces 28a-28d similar to as discussed with respect to FIG. 8. Although the light collection component 16 is depicted in FIGS. 9A and 9B as being on a same side of the substrate 28 as the reactant(s) 70, the light collection component 16 may be located at a different side of the substrate 28 relative the side at which the reactant(s) are located.

The light collection component 16 in the examples of FIGS. 9A and 9B, may include the collection lens system 36 having a first lens 36a (e.g., a cylinder lens and/or other suitable lens) configured to focus light from the reactant(s) 70 in one direction and a second lens 36b (e.g., an aspheric condensing lens and/or other suitable lens) configured to focus the light in two directions from the first lens 36a at the light collection component 16, such as a core of the optical fiber 56. In some examples, the first lens 36a may be configured to have a same focal length and diameter as the second lens 36b, but other suitable configurations are contemplated. Although not required, a mechanical aperture stop 68 may be positioned between the first lens 36a and the second lens 36b to limit an overall aperture size. A diameter of the aperture created by the mechanical aperture stop 68 may be selected to be equal to or less than a diameter of the first lens 36a and/or the second lens 36b. In some cases, a focal length of the first lens 36a, a focal length of the second lens 36b, and a diameter of the mechanical aperture stop 68 may be selected such that a numerical aperture is created that is equal to or larger than that of the optical fiber 56 to ensure optimal light collection efficiency.

In the examples of FIGS. 9A and 9B, the illumination component 12 may include a first light source 30a and a second light source 30b (not depicted in FIG. 9A) configured to provide light to a second surface 28b of the transparent substrate 28, where the first surface 28a of the transparent substrate 28 is supporting the reactants 70 of the reactant array 26 and is non-parallel with the second surface. The substrate 28 may have one or more transparent portions extending between the first surface 28a and the second surface 28b.

The light from the light sources 30 that travels through the transparent substrate 28 may be focused on the reactant(s) 70 to increase an efficiency between light used to illuminate the reactant(s) 70 and light collected from the reactant(s) (e.g., to reduce wasted light or reduce light not used to illuminate the reactant(s) 70). When the transparent substrate 28 has a semi-circular cross-section, the substrate 28 may have a focusing power in one direction such that all or at least part of the focusing of the light from the light sources 30 on the reactant(s) 70 may be achieved by the properties of the substrate 28. Additionally or alternatively, one or more lenses may be utilized to focus light on the reactant(s) 70 and/or the transparent substrate 28 may be configured in one or more manners to focus light on the reactant(s) 70.

As depicted in FIGS. 9A and 9B a porous material 64 may be coated on the first surface 28a of the transparent substrate 28 and the material of the reactants 70 may be applied to a layer of porous material 64 such that a surface area of each reactant 70 is increased relative to when the reactant 70 is applied directly to the first surface 28a. The layer of the porous material layer may have any suitable thickness T. In some examples, the thickness T of the layer of porous material 64 may be less than, equal to, or greater than a penetration depth of an evanescent wave created when light of the light source impinges on the first surface 28a (e.g., where a penetration depth and/or wavelength of the evanescent wave may be on the order of 400 nm to 1000 nm). The porous material 64 may be transparent and/or the thickness T of the layer of the porous material 64 may be set at a level that allows light to pass therethrough. The reactant(s) 70 may be applied to (e.g., printed and/or otherwise immobilized at) the layer of porous material 64 with sufficient thickness (e.g., about a length of the evanescent wave) such reactant(s) may react to fluid to be sensed that may reach a bottom of the layer of porous material 64.

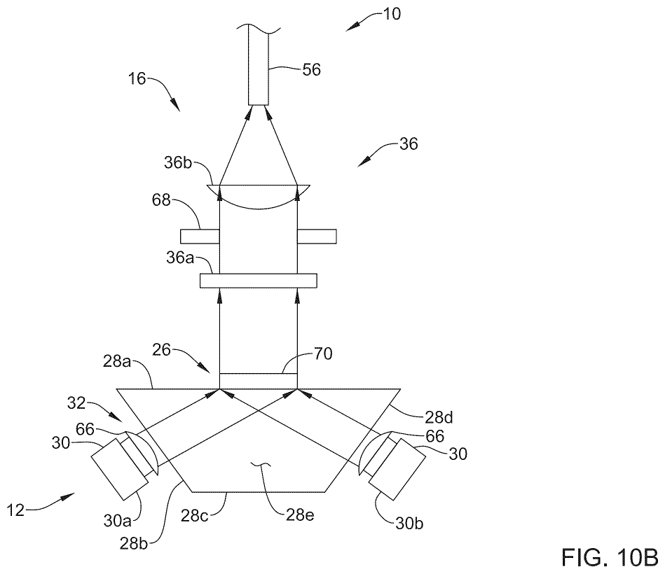

FIGS. 10A and 10B depict schematic side and end views, respectively, of a portion of the fluid analysis system 10 including the illumination component 12, the light collection component 16, and a transparent substrate 28 having a trapezoid cross-section shape having first through sixth surfaces 28a-28f similar to as discussed with respect to FIG. 8. Although the light collection component 16 is depicted in FIGS. 10A and 10B as being on a same side of the substrate 28 as the reactant(s) 70, the light collection component 16 may be located at a different side of the substrate 28 relative the side or surface at which the reactant(s) are located.

The light collection component 16 in the examples of FIGS. 10A and 10B, may include the collection lens system 36 having a first lens 36a (e.g., a focusing-in-one-direction lens or cylinder lens and/or other suitable lens) configured to focus light in one direction from the reactant(s) 70 and a second lens 36b (e.g., an imaging lens and/or other suitable lens) configured to focus the light from the first lens at a light collection component, such as a core of the optical fiber 56, and the mechanical aperture stop 68. The light collection component of FIGS. 10A and 10B may be configured the same as or similar to the configuration of the light collection component 16 of FIGS. 9A and 9B, but other suitable configurations are contemplated.

In the examples of FIGS. 10A and 10B, the illumination component 12 may include a first light source 30a and a second light source 30b configured to provide light to a second surface 28b and fourth surface 28d of the transparent substrate 28, respectively, where the first surface 28a of the transparent substrate is supporting the reactant(s) 70 of the reactant array 26 and is non-parallel with the second surface 28b and the fourth surface 28d. Additionally or alternatively, the light sources 30 may provide light to the reactant(s) 70 through one or more other surfaces of the substrate 28. The substrate 28 may have one or more transparent portions extending between the first surface 28a and one or more other surfaces thereof.

The light from the light sources 30 that travels through the transparent substrate 28 may be focused on the reactant(s) 70 to increase an efficiency between light used to illuminate the reactant(s) 70 and light collected from the reactant(s) 70. Such focusing of the light used to illuminate the reactant(s) 70 may be accomplished in transparent substrates 28 having a trapezoid cross-section section or other cross-section shape with flat outer surfaces through which light is to travel (e.g., triangle cross-section shapes, etc.) with the illumination lens system 32. The illumination lens system 32 may be configured to focus light from the light sources 30 on the reactant(s) 70 using one or more lens for each light source 30. In some examples, the illumination lens system 32 may include a focusing lens 66 that is positioned between the light source 30 and the transparent substrate 28, but other suitable configurations are contemplated. Additionally or alternatively, features of a lens may be formed into one or more sides or surfaces of the transparent substrate 28 to focus light on the reactant(s) 70 and/or the transparent substrate 28 may be configured in one or more other suitable manners to focus light on the reactant(s) 70 and/or at the light collection component 16 when light collected from the reactant(s) 70 travels through the substrate 28.

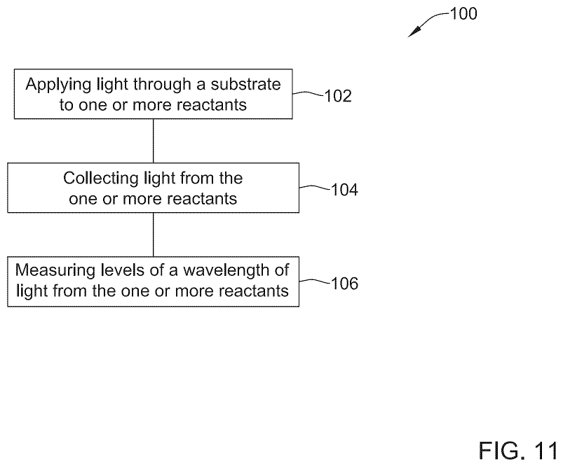

FIG. 11 depicts a method 100 that may facilitate performing a fluid analysis test on one or more fluids of interest. The method 100 may include applying 102 light from one or more light sources through one or more transparent portions of a substrate to one or more reactants on or otherwise supported by a surface of the substrate. The substrate may be transparent and/or may include a transparent portion as discussed herein or otherwise and similarly, the light may be applied to and/or through the substrate as discussed herein or otherwise. For example, although other configurations are contemplated, a first light source may apply light to the one or more reactants on a first surface of a substrate through a second surface of and a transparent portion of the substrate and a second light source may apply light to the one or more reactants through a third surface of and a transparent portion of the substrate, where the second and third surfaces may be parallel to and/or non-parallel to the first surface. In some examples, the light applied through the transparent portion(s) of the substrate may be focused by the substrate and/or by one or more lenses on a reactant of the one or more reactants. Further, before and/or during the application of light to the substrate, the one or more reactants on or otherwise supported by the substrate may be exposed to a fluid to be tested during the fluid analysis test.

The method 100 may include collecting 104 light from the one or more reactants. Although not required, the light may be collected from the one or more reactants while the light is being applied to the one or more reactants through the transparent portion of the substrate. Further, the light collected from the one or more reactants may be collected from a same side of the substrate as a side at which the one or more reactants are located and/or from a side of the substrate that is different than the side at which the one or more reactants are located and through a transparent portion of the substrate 28, as discussed herein or otherwise.

The method 100 may include measuring 106 levels of a wavelength of light collected from the one or more reactants. The levels of the wavelengths of light collected from the one or more reactants may be measured in any suitable manners as discussed herein or otherwise and including, but not limited to, by counting photons at one or more wavelengths of light collected, measuring an amount of light collected at one or more wavelengths of light collected, a change in photon count over time for one or more wavelengths of light collected, a change in pixel value (e.g., a change in pixel grayscale value) of an image sensor over time, and/or levels of wavelengths of light collected may be measured in one or more other suitable manners.

The measurements of the levels of the wavelengths of light collected from the one or more reactants may be utilized to identify a component of the fluid to which the one or more reactants may be exposed. In some examples, when the levels of the wavelengths of light collected over time match or closely resemble a set of expected wavelength levels of the reactant(s) exposed to a known fluid or component of fluid, the known fluid or component of fluid may be identified as the fluid or as a component of the fluid tested in the fluid analysis test. Example techniques for measuring levels of wavelength of light collected from one or more reactants and for comparing measurements to known measurements associated with fluids are discussed in PCT Patent Application No. PCT/US2023/083024 (Attorney docket no. 1519.1004111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system comprising:
a substrate having a transparent portion;
one or more reactants on a first surface of the transparent portion of the substrate;
a light source; and
a light collector, and
wherein the light source is configured to illuminate the one or more reactants through the transparent portion of the substrate and the light collector is configured to collect light from the one or more reactants.

2. The system of claim 1, wherein the transparent portion of the substrate extends between the first surface and a second surface of the substrate, wherein the second surface of the substrate is non-parallel with the first surface.

3. The system of claim 2, wherein the light source is configured to illuminate the one or more reactants by applying light to the one or more reactants through the second surface.

4. The system of claim 2, wherein the second surface is rounded.

5. The system of claim 2, further comprising:
a lens positioned between the light source and the second surface, and
wherein the lens is configured to focus light from the light source on a reactant of the one or more reactants.

6. The system of claim 1, wherein a cross-section of the substrate is symmetrical about a center line extending perpendicularly through the first surface.

7. The system of claim 1, wherein a shape of a cross-section of the substrate is selected from a group consisting of a trapezoid, a semi-circle, and a triangle.

8. The system of claim 1, further comprising:
a collection lens system, wherein the collection lens system is configured to focus light from the one or more reactants for collection by the light collector.

9. The system of claim 8, wherein the collection lens system comprises a cylinder lens and an aspheric lens.

10. The system of claim 1, further comprising:
an optical fiber configured to collect light from the one or more reactants.

11. The system of claim 1, wherein the light collector comprises one or more light sensors configured to receive light from the one or more reactants and the one or more light sensors are selected from a group consisting of a spectrometer, a contact imaging sensor, a camera, and an n-dimensional sensor array.

12. The system of claim 1, further comprising:
a layer of porous material on the first surface of the substrate, and
wherein the one or more reactants are on the layer of porous material.

13. A method comprising:
applying light through one or more transparent portions of a substrate to one or more reactants on the substrate;
collecting light from the one or more reactants; and
measuring levels of a wavelength of the light from the one or more reactants.

14. The method of claim 13, further comprising:
exposing the one or more reactants to a fluid; and
identifying a component of the fluid based on the levels of the wavelength of the light from the one or more reactants.

15. The method of claim 14, wherein the collecting light from the one or more reactants comprises collecting light from the one or more reactants through the one or more transparent portions of the substrate.

16. The method of claim 13, wherein the applying the light through the one or more transparent portions of the substrate to the one or more reactants includes focusing the light on a reactant of the one or more reactants.

17. The method of claim 13, wherein a transparent portion of the one or more transparent portions of the substrate extends between a first surface on which the one or more reactants are located and a second surface of the substrate, wherein the second surface of the substrate is non-parallel with the first surface.

18. A colorimetric sensor array comprising:
a substrate having a transparent portion;
one or more reactants on a first surface of the transparent portion the substrate, and
wherein the transparent portion of the substrate extends between the first surface on which the one or more reactants are located and a second surface of the substrate, wherein the second surface of the substrate is non-parallel with the first surface.

19. The colorimetric sensor array of claim 18, wherein the second surface is rounded.

20. The colorimetric sensor array of claim 18, wherein a cross-section of the substrate is symmetrical about a center line extending perpendicularly through the first surface.
